(19) United States
(12) Patent Application Publication
(10) Pub. No.: US 2011/0027205 A1
(43) Pub. Date: Feb. 3, 2011

(54) USE OF MYRTLE EXTRACT AS DEPIGMENTING AGENT

(75) Inventor: Bernard Fabre, Belberaud (FR)

Correspondence Address: BIRCH STEWART KOLASCH & BIRCH, PO BOX 747, FALLS CHURCH, VA, 22040-0747, US

(73) Assignee: PIERRE FABRE DERMO-COSMETIQUE, Boulogne-Billancourt (FR)

(21) Appl. No.: 12/937,483

(22) PCT Filed: Apr. 16, 2009
(86) PCT No.: PCT/EP2009/054487

§ 371 Date: Oct. 12, 2010

(30) Foreign Application Priority Data
Apr. 16, 2008 (FR) 0852570

(51) Int. Cl. A61K 8/96; A61Q 19/02; A61Q 5/00; A61Q 19/08

(52) U.S. Cl. 424/62

(57) ABSTRACT

The present invention relates to novel compositions, such as cosmetic compositions, containing a myrtle extract, and their uses for depigmenting purposes.

The present invention relates to novel compositions such as cosmetic compositions containing a myrtle extract, and their uses with respect to depigmentation.

The invention also relates to a cosmetic method for whitening and/or lightening the skin and/or body hair and/or head hair, and a cosmetic method to reduce and/or eliminate and/or prevent skin pigment marks, comprising the application to the skin and/or body hair and/or head hair of a cosmetic composition according to the invention.

### First:

Pigmentation of the skin is due to the ability of some specialized cells, the melanocytes, to synthesize melanin. Melanin is a pigment which can assume different molecular forms, melanin and its derivatives—brown black pigments, phaeomelanin and its derivatives—yellow-red pigments. The melanocytes are located in the basal layers of the epidermis, they have cytoplasmic or dendritic extensions which are needed for the transport of melanin. Melanin is in the form of particles contained in melanosomas. It is synthesized therein from tyrosine through the action of one enzyme in particular: tyrosinase. The formed melanosomas migrate into the keratinocytes via the dendritic extensions, then enter into these cells by means of a translocation phenomenon. As and when the keratinocytes differentiate as corneocytes, the melanin disperses in the cytoplasm and is eliminated by desquamation.

Tyrosinase inhibition is an important target for products which are intended to have depigmenting activity. With this inhibition melanogenesis is blocked. This is the case for numerous active ingredients such as arbutin or hydroquinone. This activity can be observed in vitro directly on the enzyme, or more globally on melanocytes whose capacity to synthesize melanin is evaluated. The ability to prevent the transport of melanosomas is also a target of interest for a depigmenting product. This can be observed by a product's ability or inability to prevent the formation of the dendrites that are essential for the migration of these melanosomas and hence of their constituent melanin.

### Second:

Myrtle, or Myrtus communis, belongs to the family of Myrtaceae.

It is a shrub which can grow 2 to 3 metres high, its stems are irregular and are covered by almost smooth, russet-coloured bark.

The leaves have opposite arrangement, are evergreen, tough, dark green. The limb is oval, sharp at the top, with translucent glandulous dots, subsiding into a very short petiole.

The flowers are white, scented and solitary carried by a long peduncle at the leaf axil. They flower from May to July. The calyx is welded to the ovary in its lower part. It has 5 triangular divisions that are outspread and shorter than the petals. The corolla has 5 petals unattached. The stamens are free and numerous. The ovary is triocular and surmounted by a style ending in a single stigma. The 3 housings of the ovary each enclose numerous ovules. The fruit is a globose berry, ovoid-shaped, bluish-black when ripe. It is surmounted by the 5 evergreen teeth of the calyx. It encloses a large number of albumin-free seeds.

Myrtle grows indifferently in chalky or siliceous soil. It is not frost-resistant and cannot therefore be grown at high altitudes. It is very common in France along the Mediterranean coast, especially in Provence. It is also native to Southern Europe, Western and Southern regions of Asia, North Africa.

Myrtle is especially used for its high essential oil content in the leaves, but also in the fruit. This essence which can be extracted by steam distillation or using apolar solvents such as hexane, is of greenish colour with a pleasant smell. It consists of eucalyptol, linalol, geraniol, pinene and limonene, and more specific substances such as myrtol and myrtenol.

The leaves also contain high proportions of tannins, chiefly gallic tannins, 14% on average, and also flavonoids (myricetol, kaempferol and their glycosilated derivatives), coumarins (aesculine, aesculetine) and phenol acids.

The fruits, in addition to essential oils, contain a large quantity of polyphenol compounds of which part consists of hydrolysable, condensed tannins. Simpler phenol compounds are also found such as quercetin, patuletin, gallic and ellagic acids.

Traditionally, myrtle is used for its essential oil. For example G. GARNIER et al in their book: Ressources Médicinales de la Flore Francaise, published by Vigot Frères in Paris in 1961, describe that << The essential oil, formerly administered under the inappropriate name myrtol, endowed with antiseptic and disinfecting properties, acts as digestion stimulant, is haemostatic and, in addition, has a marked action on squamous skin disorders, and chiefly on psoriasis >>. The indication for psoriasis is also described in patent FR 2 783 425 titled << Myrtle extract containing myrtucommulone B′, its method of preparation and applications in dermatology and cosmetology >>.

The use as deodorant of a composition containing essential oil of myrtle, in association with other plants, was also the subject of patent application US2001006626.

Patent application JP2001220312 describes a hydrating distilled water containing essential myrtle oil.

Again in cosmetology, but as hair product, extracts of myrtle either alone or in association, have been the subject of anti-dandruff studies: << Hair composition comprising a myrtle extract, its method of preparation and particular use for anti-dandruff treatment: FR 2 735 026, and << Association of myrtle extract and anti-fungal >>: FR 2 741 265.

The present invention proposes a novel depigmenting active ingredient derived from a plant extract: myrtle or Myrtus communis. In fully surprising and innovative manner, the Applicant has evidenced that a myrtle extract is capable of inhibiting the synthesis and transport of melanin by the melanocytes.

The present invention relates to the use of a myrtle extract in a cosmetic composition as depigmenting agent, and also relates to a cosmetic whitening and/or lightening method of the skin and/or body hair and/or head hair, and a cosmetic method to reduce and/or eliminate and/or prevent skin pigment marks, comprising the application to the skin and/or body hair and/or head hair of a cosmetic composition containing a myrtle extract.

A further subject of the present invention is the use of a myrtle extract to produce a dermatological composition intended to depigment the skin.

Finally, the object of the invention is to provide novel compositions, such as cosmetic or dermatological compositions which, as active ingredient, contain a myrtle extract in a physiologically acceptable medium, advantageously in association with another active ingredient such as ascorbyl glucoside.

Under the present invention, the myrtle extract can be prepared following conventional extraction and concentration steps known to persons killed in the art.

The myrtle extract can be obtained from the whole plant or part of the plant. In particularly advantageous manner according to the present invention, the myrtle extract is obtained from the leaves, the fruit, or a mixture of myrtle leaves and fruit.

Therefore, the production of a myrtle extract from the leaves and/or fruit of myrtle can comprise the following steps:
grinding the myrtle leaves and/or fruit,
extracting the ground myrtle leaves and/or fruit using a solvent such as a polar solvent,
collecting the extraction solution by filtering or spinning,
concentrating the collected solution by evaporation, to obtain the myrtle extract which is optionally to be mixed with other constituents to obtain said composition.

Preferably, the myrtle leaves and/or fruit are dry myrtle leaves and/or fruit.

Further preferably the weight ratio of the myrtle leaves to the fruit is close to 90/10.

During the extraction step, the solvent used is advantageously a polar solvent such as a C1 to C4 alcohol or acetone, or a water-alcohol mixture or an acetone-water mixture.

The weight ratio of the leaves and/or fruit to the solvent is typically between 1 and 20, preferably between 1 and 4, and the extraction temperature is generally between room temperature and the boiling point of the solvent. The extraction time is typically between 1 hour and 24 hours.

According to one advantageous embodiment of the invention, the concentration of the solution collected by evaporation is obtained under reduced pressure, at a temperature of between 40 and 100° C., until a powder is obtained. It is also possible, during the concentration operation, to add a solvent with high boiling point and more particularly glycerine, propylene glycol, butylene glycol or transcutol. The myrtle extract is then in liquid form.

Advantageously, the myrtle extract comprises polyphenols in a quantity of between 1 and 50%, preferably between 10 and 40%, and further preferably between 20 and 30 dry weight %, relative to the dry matter of the extract.

Amongst the polyphenols, flavonoids are typically found as well as tannins.

According to one advantageous embodiment of the invention, the myrtle extract comprises flavonoids in a quantity of between 0.1 and 5%, preferably between 1 and 4% and further preferably between 2 and 3 dry weight %, relative to the dry matter of the extract.

According to another advantageous embodiment of the invention, the myrtle extract contains tannins in a quantity of between 1 and 50%, preferably between 5 and 40%, and further preferably between 10 and 30% dry weight, relative to the dry matter of the extract.

According to one particular characteristics of the present invention, the myrtle extract contains flavonoids in a quantity of between 0.5 and 4%, typically between 1 and 3.5%, and tannins in a quantity of between 5 and 40%, typically between 10 and 40%, for example between 20 and 40%.

One of the subjects of the present invention relates to the use of a myrtle extract as depigmenting agent in a cosmetic composition.

Advantageously, the cosmetic composition of the invention is intended to whiten and/or lighten the skin and/or body hair and/or head hair.

The myrtle extract according to the present invention can be used to obtain uniform complexion; which is characterized by even skin tone, that is lighter, brighter and more transparent. Skin radiance is therefore improved.

The advantages obtained with the composition of the present invention are of particular interest for sensitive skins, irrespective of type (dry, normal, oily), and more particularly for lifeless sensitive skins lacking radiance.

Myrtle extract as depigmenting agent has also shown good capacities:
to attenuate visibly the intensity and size of pigment marks;
to regulate and/or inhibit the production of melanin, responsible for pigmentation;
to dim visible marks;
to prevent the onset of additional marks;

demonstrating its advantage for the depigmenting of certain unsightly pigment marks due to epidermal hyperpigmentation; such as age spots of the skin in particular.

A further subject of the present invention is the use of a myrtle extract to produce a composition such as a cosmetic or dermatological composition intended to depigment the skin and/or body hair and/or head hair.

With the use of a myrtle extract according to the invention, it is advantageously possible:
to reduce and/or eliminate pigmentation marks, such as hyperpigmentation marks due to pro-inflammatory stress, e.g. brownish pigment marks induced by UVs, or to reduce and/or eliminate chloasmas;
to reduce and/or inhibit the production of melanin, responsible for pigmentation.

The myrtle extract according to the present invention can therefore advantageously be used in a composition, such as cosmetic composition, to reduce and/or eliminate skin aging spots, or to reduce and/or eliminate brownish pigment marks which may be induced by UVs, or chloasmas.

The invention also relates to cosmetic and/or dermatological compositions characterized in that, as active ingredient, they contain a myrtle extract in a physiologically acceptable medium, i.e. compatible with the skin and/or scalp, mucous membrane, head hair, body hair and/or eyes.

Preferably the cosmetic or dermatological composition of the present invention contains a quantity of myrtle extract, as active ingredient, of between 1 mg and 50 g, preferably between 10 mg and 10 g per 100 g of said composition.

Advantageously, said quantity of myrtle extract lies between 50 mg and 1 g per 100 g of composition. Further advantageously, said quantity of myrtle extract lies between 100 mg and 500 mg per 100 g of composition.

Preferably, said compositions also contain at least one second depigmenting active ingredient.

Said other agent or agents which can be added to the present composition are known to those skilled in the art, who are capable of adjusting the relative proportions of each constituent of the composition in order to optimise the efficacy of said composition.

Advantageously, mention may be made by way of indication and in non-limiting fashion of the active ingredients chosen from the group formed by vitamin C and its derivatives including ascorbyl glucoside, salicylic acid, lactic acid, glycolic acid, malic acid, arbutin, kojic acid, vitamin B3, linoleic acid and vegetable oils containing the same, retinaldehyde and its derivatives, resorcinol and plant extracts in particular bearberry, liquorice, mulberry, arbutus, Broussonetia, camomile or Skutellaria.

Preferably, a composition according to the invention comprises a myrtle extract (Myrtus communis) and at least one depigmenting active ingredient chosen from the group formed by ascorbyl glucoside, lactic acid, glycolic acid, malic acid, arbutin, kojic acid, linoleic acid and vegetable oils containing the same, retinaldehyde and its derivatives, resorcinol and plant extracts in particular bearberry, liquorice, mulberry, arbutus, Broussonetia, camomile or Skutellaria, used alone or in a mixture.

More preferably, a composition according to the invention comprises a myrtle extract (Myrtus communis) and at least one depigmenting active ingredient chosen from the group formed by ascorbyl glucoside, glycolic acid, malic acid, arbutin, kojic acid, linoleic acid and vegetable oils containing the same, retinaldehyde and its derivatives, and plant extracts in particular bearberry, liquorice, mulberry, arbutus, Broussonetia or Skutellaria, used alone or in a mixture.

In particularly advantageous manner, the composition of the present invention comprises a myrtle extract in association with ascorbyl glucoside.

The compositions of the invention which contain a myrtle extract in association with at least one other depigmenting active ingredient, are advantageously used in all the above-mentioned applications.

The cosmetic and/or dermatological composition of the present invention can advantageously have any galenic form normally used in the areas of cosmetics and dermatology for topical or oral application.

Preferably, the topical form can be in the following particular form:
an aqueous hydro-alcohol solution, optionally gelled,
a dispersion of lotion type, optionally a two-phase dispersion,
an oil-in-water or water-in-oil emulsion, or multiple,
an aqueous gel,

and can have the appearance of a serum, cream, gel, ointment, milk, lotion, paste or foam. It can also be applied in spray form or in solid form e.g. in stick form.

One of the advantages of the present invention lies in the fact that the compositions of the invention have good skin tolerance, even for sensitive skins, irrespective of type (dry, normal, oily).

This composition can also be in oral form, such as tablets, capsules, powders for drinkable suspension.

The composition can also contain all the constituents usually used in the application under consideration. Particular mention can be made of water, solvents, mineral oils, animal and/or vegetable oils, waxes, pigments, chemical or mineral filters, anti-oxidants, fillers, surfactants, stabilisers, preserving agents, perfumes and colouring agents.

The choice and/or quantity of the ingredient(s) is also determined by the specific needs of the skin and/or body hair and/or head hair to which the composition is to be applied, and by the properties and desired consistency of the composition according to the present invention.

The invention will be better understood with the following non-limiting examples describing particular embodiments of the cosmetic and/or dermatological compositions of the invention.

### EXAMPLES 1-4

### Preparation of a Myrtle Extract

### Example 1

100 kg of myrtle leaves and dry fruit are ground then extracted with 700 kg of 50% ethanol. Extraction is conducted under reflux and stirring for 1 hour. The solutions are collected after filtration, then concentrated under reduced pressure at 60° C. Concentration then drying are conducted until a dry matter is obtained whose polyphenol content is between 20 and 40% and flavonoid content is between 1.5 and 3.5%.

### Example 2

The first steps are identical to those in example 1, but procedure is different for the last concentration and drying steps. After extraction and during concentration 200 kg propylene glycol are added, and concentration is continued until a solution weighing 220 kg is obtained. The dry matter content lies between 8 and 12%, that of the polyphenols relative to the dry matter is between 20 and 40% and the flavonoid content relative to the dry matter is between 1.5 and 3.5%.

### Example 3

The starting raw material in this example is different to the material in example 1. Also another extraction solvent is used to obtain an extract having different flavonoid and polyphenol contents.

1 kg of dry, ground leaves are cold extracted with 10 kg methanol for 12 hours. The methanol solution is vacuum concentrated at 60° C., then dried. The dry extract obtained is ground. The powder contains between 30 to 40% polyphenols and between 3 and 4% flavonoids.

### Example 4

10 kg of ground leaves and fruit are extracted with a water and ethanol mixture in proportions of 80 to 20, under reflux and stirring for 3 hours. The solution obtained is spun, then filtered.

Its polyphenol content relative to the dry matter lies between 5 and 10%. The flavonoid content varies between 0.5 and 1.5%.

### Example 5

### Evaluation of Depigmenting Activity

### 5.1 Activity on the Dendricity of Culture Melanocytes

Skin pigmentation results from interactions between epidermal melanocytes and keratinocytes. The dendrites of the melanocytes play an important part in the pigmentation process. They allow the transfer of melanosomas into the basal and supra-basal parts of the epidermis. They consist of an association of microtubules and actin filaments which enable the melanin particles to move from inside the melanocytes towards the tips of the dendrites, and their distribution on the skin (Hara et al., J Invest Dermatol, 2000).

Differentiation of the melanocytes translates as stimulation of melanogenesis and dendricity. This process is induced by several cAMP-inducing factors: α-MSH, Forskoline (Busca et al, 2000).

Direct morphological assessment of the effect of depigmenting or pigmentation-stimulating agents on changes in melanocyte dendricity is performed by marking the actin filaments of the melanocytes with phalloidin-rhodamine.

In this study the Applicant evaluated the effect of myrtle extract on changes in dendricity of melanocytes which were or were not pre-treated with α-MSH. The reference depigmenting agent used was the Kligmann trio.

This agent consists of a mixture of 3 active ingredients, hydroquinone (1%), hydrocortisone (0.5%) and retinoic acid (0.0125%). For the test, the suspensions of melanocytes are placed on glass slides for adhesion, then treated or non-treated for one hour with αMSH and exposed to different concentrations of myrtle extract such as defined in example 1 for one hour. The cells are then treated with a staining developing agent, phalloidin-TRITC to reveal their actin network and their dendricity, and they are then observed under microscope to examine their morphology.

The melanocytes treated solely with αMSH show an increase in their melanocyte dendricity compared with the non-treated cells, thereby exhibiting the propigmenting effect of αMSH. On the other hand, the melanocytes pre-treated with αMSH then with the Kligmann trio diluted to 1/75000 or pre-treated with a myrtle extract of 0.3 μg/ml or 1 μg/ml do not show any increase, or only a very slight increase, in their dendricity.

These 2 substances therefore display an αMSH-inhibitor effect, an endogenous factor for stimulation of melanin biosynthesis, and hence their capacity to exhibit depigmenting activity.

### 5.2 Activity on Melanogenesis

The melanocytes are left to incubate in a suitable culture medium at 37° C. for 72 hours in contact with the myrtle extract according to example 1, at different concentrations, and with hydroquinone, the reference substance. At the end of the incubation period, the melanocytes are collected and washed. The cells are then treated with a sodium solution to extract the melanin. The stained solutions are then assayed under 450 nm spectrophotometry for their melanin content using a calibration curve. A melanogenesis-inhibiting activity of the tested products will translate as a lower melanin content in the extracted melanocyte solutions. A prior cytotoxicity test is evidently performed on these same cells to verify the non-toxicity of the extracts at the tested concentrations.

The inhibiting activity of melanin synthesis displayed by the different tested products is given in table 1 below.

TABLE 1
evaluation of activity on the melanogenesis
of a myrtle extract according to the invention

| | Concentrations | |
|---|---|---|
| Products | tested | % inhibition |
| Control | — | — |

| | | | |
|---|---|---|---|
| Hydroquinone | 0.005 | mM | 35.7 |
| | 0.01 | mM | 58 |
| Myrtle | 5 | μg/ml | 41.7 |
| extract | 10 | μg/ml | 43.7 |
| | 25 | μg/ml | 39.4 |

Hydroquinone effectively exhibits an inhibiting activity of melanin content and hence of melanogenesis at the tested concentrations, and thereby validates the activity evaluation test.

The myrtle extract shows inhibiting activity of melanogenesis at the 3 tested concentrations, and therefore shows the interest of the product for depigmentation.

### Example 6

### Cosmetic Serum

| Compound | Quantity (/100 g) |
|---|---|
| Myrtus communis dry extract | 0.1 g to 0.5 g |
| (example 2) | |
| Ascorbyl glucoside | 1.0 g to 10.0 g |
| Cyclomethicone | 2.0 g to 10.0 g |
| Carbomer | 0.5 g to 0.6 g |
| Propylene glycol | 1.0 g to 5.0 g |
| Glycerine | 1.0 g to 5.0 g |
| Triethanolamine | 0.5 g to 2.5 g |
| Purified water | To complete up to: 100 g |

### Example 7

### Study of the Efficacy of the Serum According to Example 6

### First Trial

The clinical trial given below was an open trial conducted on 30 Japanese women aged 25 to 45 years:
all having sensitive skin (100%)
and having pigmentation marks on the face, at least one mark having a diameter of more than 5 mm.

Amongst this cohort, different skin types were represented:
normal skin (17%)
mixed/oily skin (26%)
oily skin (23%)
mixed/dry skin (17%)
dry skin (17%).

This trial concerned the serum composition given previously under example 6. D0 was the starting date of the trial (before the first application of the serum). The product was applied twice a day, morning and evening, for 12 weeks—from D1 to D84) over the entire face and neck, paying particular attention to the pigmentation marks.

This cosmetic composition was applied instead of the usual hydrating product.

Different evaluation techniques were used:

1. Colorimetry/Measurement Parameter for Skin Pigmentation: Melanic Index

The melanic index of the skin was measured using a MEXAMETER® MX16 (Courage and Khazaka).

The mexameter probe emits radiation in 3 wavelengths over the area to be measured, which is absorbed by the skin. The diameter of the measurement surface is 5 mm.

A receiver then measures the light reflected by the skin. Two wavelengths are chosen to measure melanin.

The apparatus then indicates a melanic index in arbitrary units (a.u.) in relation to the quantity of melanin in the skin at the point of measurement.

Protocol

Measurement areas: the melanic index of the skin was measured at the mark having a diameter of more than 5 mm, and in a healthy area in the vicinity of said mark.

Measurement times: the values were read at D0 (before the first application of the serum) and at D42 (after 42 days' treatment) and D84 (after 84 days' treatment).

Results

The results of the measurements taken at each measurement time and in each individual were calculated as a mean per area (hyperpigmented area, healthy skin area).

Δ (expressed in arbitrary units (a.u.)) denotes the difference between the melanic index of the hyperpigmented area and the melanic index of the healthy area.

Table 2 below gives the results obtained:

TABLE 2
melanic indexes of the skin treated with the
serum containing myrtle extract

| | Δ melanic index |
|---|---|
| Measurement | (in a.u.) |
| times | (measurement mean) |
| D0 | 25.5 ± 2.9 |
| D42 | 23.6 ± 2.9 |
| D42 − D0 (as %) | −7.5% |
| D84 | 23.0 ± 3.1 |
| D84 − D0 (as %) | −9% |

Over the 12 weeks of treatment, it is observed that the value of Δ is significantly reduced compared with its initial value at D0: reduction of 9%. This reduction is observed as early as 6 weeks after start of treatment (D42) when a Δ reduction of 7.5% was found.

This reasonably translates as a tendency of the tested composition to reduce the melanic index of the pigment marks.

The Applicant has thereby evidenced that application of a composition according to the present invention induces a significant homogenizing effect of the complexion.

2. Clinical Assessment

A Dermatologist ascertained the initial condition of the skin of each person entering the trial (D0); then followed up the skin condition after 28 days and after 84 days of treatment.

The examined parameters were the following:
aspect of the pigment marks,
skin radiance,
skin lightness, and
even skin tone.

Protocol

The different parameters were examined in accordance with the 9-point << Clinical storing system >>:

| SLIGHT | | MODERATE | | MAJOR | |
|---|---|---|---|---|---|
| + | 1 | + | 4 | + | 7 |
| ++ | 2 | ++ | 5 | ++ | 8 |
| +++ | 3 | +++ | 6 | +++ | 9 |

Results

Δ₂₈ and Δ₈₄ are used to denote the variations (expressed as %) in each criterion at D28 and D84 respectively, relative to their value at D0.

At D0, D28 and D84, the assessment of the criteria was as follows (Table 3):

TABLE 3
clinical trial on 4 parameters led by a Dermatologist on 30 women

| Examined | value at | value at | | value at | |
|---|---|---|---|---|---|
| parameter: | D 0 | D 28 | Δ₂₈ | D 84 | Δ₈₄ |
| aspect of marks | 5.2 ± 1.5 | 4.8 ± 1.6 | −8% | 3.9 ± 1.4 | −25% |
| skin radiance | 5.6 ± 1.9 | 6.7 ± 1.5 | +20% | 7.3 ± 1.3 | +30% |
| skin lightness | 5.6 ± 1.8 | 6.3 ± 1.4 | +13% | 6.9 ± 1.6 | +23% |
| even skin tone | 5.8 ± 1.8 | 6.4 ± 1.4 | +10% | 6.9 ± 1.6 | +19% |

After 4 weeks (D28), and advantageously after 12 weeks (D84) a favourable, significant variation in all assessed criteria was observed: i.e. less apparent marks and a more radiant, lighter and even skin tone.

Conclusion of the First Efficacy Trial

This trial was able to show the significant efficacy of a composition containing a myrtle extract according to the present invention for sensitive skins of different types having pigment marks. The benefits obtained translate as less visible marks and improved complexion in terms of lightness, radiance and uniformity, and homogeneity.

### Second Trial

This second clinical trial described below was a blind trial conducted on 40 Japanese individuals (37 women and 3 men) aged 22 to 48 years.

This trial concerned the serum composition given previously under example 6.

The starting date of the trial is denoted D0 (before the first application of the serum). The product was applied once a day in the evening after washing, for 3 months (from D1 to D90) on the inner surface of the forearm (chosen at random).

Evaluation Technique Used: Colorimetry/Parameter for Measurement of Skin Pigmentation: Change in Parameter B*

Parameter b* (blue-yellow axis) of colorimetry was measured using a CR 400® chromameter (Minolta).

The probe of the chromameter emits a flash of white light on the area to be measured, which is absorbed by the skin. The diameter of the measured surface is 8 mm. Three sensors, corresponding to the cones of the human eye, then record the light reflected by the skin. The apparatus indicates values L*, a* and b*, in arbitrary units (a.u.). These values relate to the quantity of melanin in the skin at the point of measurement.

Protocol

Measurement areas: skin pigmentation was measured on the inner surface of the forearm (treated area), and at an adjacent non-treated area (control area)—these measurement areas were previously delimited and identified with identification masks.

Measurement times: the values are read at D0 (before the first application of the serum), at D30 (after 1 month's treatment), D60 (after two months' treatment) and D90 (after 3 months' treatment).

Results

The results of the measurements taken at each measurement time and in each person were calculated as a mean per area (treated area, control area).

To evaluate the changes in skin colour subsequent to application of the serum according to the present invention, for each measurement time (D30, D60, D90) the value of b* is expressed for each area, and the difference between the colorimetric values of the treated area compared with the control area.

TABLE 4
colorimetric parameter b* of the skin area
treated with the serum containing a myrtle extract, and
of the adjacent control area

| | | mean b* | | % trend in |
|---|---|---|---|---|
| | mean b* | (in | | mean values |
| | (in a.u.) | a.u.) | | (b*_{treated area} − |
| Measurement | treated | control | b*_{treated area} − | b*_{control area)} |
| time | area | area | b*_{control area} | relative to D0 |
| D0 | 18.52 | 17.18 | 1.34 | — |
| D30 | 18.20 | 16.97 | 1.23 | −18.7% |
| D60 | 17.25 | 15.78 | 1.47 | +4.5% |
| D90 | 16.96 | 16.28 | 0.68 | −44.6% |

First, it is observed that value b* of the treated area reduces progressively and distinctly throughout the period of the trial up until D90, which translates as lightening of the treated area.

Second, after 3 months' treatment (D90) a significant reduction is observed compared with D0 for the difference (b*_{treated} area−b*_{control} area): i.e. a 44.6% reduction in this difference. This reasonably demonstrates a tendency of the tested composition to reduce skin pigmentation.

Conclusion of the Second Efficacy Trial

The Applicant has evidenced that the application of a composition according to the present invention has a depigmenting effect.

1.-14. (canceled)

15. Cosmetic use of a myrtle extract (Myrtus communis) in a cosmetic composition as depigmenting agent.

16. Use according to claim 15, characterized in that the composition is intended to whiten and/or lighten the skin and/or body hair and/or head hair.

17. Use according to claim 15, characterized in that the composition is intended to reduce and/or eliminate skin age spots.

18. Use according to claim 15, characterized in that the composition is intended to reduce and/or eliminate brownish pigment marks which may be induced by UVs, or chloasmas.

19. Use according to claim 15, characterized in that the quantity of myrtle extract lies between 1 mg and 50 g per 100 g of composition.

20. Use according to claim 15, characterized in that the composition at least one other depigmenting agent in a physiologically acceptable medium.

21. Use according to claim 20, characterized in that the other depigmenting agent is chosen from the group consisting of vitamin C and its derivatives such as ascorbyl glucoside, salicylic acid, lactic acid, glycolic acid, malic acid, arbutin, kojic acid, vitamin B3, linoleic acid and vegetable oils containing the same, retinaldehyde and its derivatives, resorcinol, bearberry extract, liquorice extract, mulberry extract, arbutus extract, Broussonetia extract, camomile extract and Scutellaria extract, used alone or in a mixture.

22. Cosmetic or dermatological composition which, as depigmenting active ingredient, contains a myrtle extract (Myrtus communis) and also in a physiologically acceptable medium at least one other depigmenting agent chosen from the group consisting of ascorbyl glucoside, lactic acid, glycolic acid, malic acid, arbutin, kojic acid, linoleic acid and vegetable oils containing the same, retinaldehyde and its derivatives, resorcinol, bearberry extract, liquorice extract, mulberry extract, arbutus extract, Broussonetia extract, camomile extract and Scutellaria extract, used alone or in a mixture.

23. Composition according to claim 22, wherein the depigmenting agent is chosen from the group consisting of ascorbyl glucoside, glycolic acid, malic acid, arbutin, kojic acid, linoleic acid and vegetable oils containing the same, retinaldehyde and its derivatives, bearberry extract, liquorice extract, mulberry extract, arbutus extract, Broussonetia extract and Scutellaria extract, used alone or in a mixture.

24. Composition according to claim 23, wherein the depigmenting agent is ascorbyl glucoside.

25. Composition according to claim 22, characterized in that the quantity of myrtle extract lies between 1 mg and 50 g per 100 g of composition.

26. A method of treatment comprising the topical administration of a dermatological composition according to claim 22 to a patient in need thereof.

27. A method of treatment according to claim 26 for depigmenting skin.

28. Cosmetic method for whitening and/or lightening the skin and/or body hair and/or head hair comprising application to the skin and/or body hair and/or head hair of a cosmetic composition containing a myrtle extract (Myrtus communis).

29. Cosmetic method to reduce and/or eliminate and/or prevent pigment marks of the skin comprising application to the skin of a cosmetic composition containing a myrtle extract (Myrtus communis).

30. Cosmetic method according to claim 28 or 29, characterized in that the composition is such as defined in claim 22.
